(19) United States
(12) Patent Application Publication
(10) Pub. No.: US 2024/0180572 A1
(43) Pub. Date: Jun. 6, 2024

(54) CLOT REMOVAL DEVICE WITH RADIOPAQUE INDICATOR

(71) Applicant: Neuravi Limited, Galway (IE)

(72) Inventors: Anushree DWIVEDI, Galway (IE); Declan LEE, Galway (IE); Sarah JOHNSON, Galway (IE)

(73) Assignee: Neuravi Limited, Galway (IE)

(21) Appl. No.: 18/073,297

(22) Filed: Dec. 1, 2022

(51) Int. Cl. A61B 17/221

(52) U.S. Cl. CPC A61B 17/221; A61B 2017/22034

(57) ABSTRACT

A clot removal assembly that includes a delivery microcatheter, a cage structure configured to transform between a delivery configuration and an expanded pinching configuration, and a shaft in communication with the cage structure and substantially disposed within the delivery microcatheter is presented herein. The catheter includes a distal portion including an elongate radiopaque indicator, and a proximal portion, and the elongate radiopaque indicator is configured to transform between a substantially straight non-indicating state and a substantially wavy indicating state responsive to an impingement of the cage structure on the delivery microcatheter.

### FIELD

The present disclosure generally relates to devices and methods for removing blockages from blood vessels during intravascular medical treatments.

### BACKGROUND

Clot retrieval devices are used in mechanical thrombectomy for endovascular intervention, often in cases where patients are suffering from conditions such as acute ischemic stroke AIS, myocardial infarction MI, and pulmonary embolism PE.

The tortuosity is a challenge in the arteries approaching the brain. For example, it is not unusual at the distal end of the internal carotid artery that the device will have to navigate a vessel segment with a 1800 bend, a 900 bend and a 3600 bend in quick succession over a few centimeters of vessel. This poses a challenge to catheters that must have sufficient column strength to transmit forces applied by the user while also having sufficient flexibility to navigate tortuous distal vessels.

The clot may have any of a range of morphologies and consistencies. For example, clot can be difficult to grip, and improper grip can lead to fragmentation which may cause embolization. Compression of a blood clot causes dehydration of the clot and results in a dramatic increase in both clot stiffness and coefficient of friction which necessitates removal by pinching rather than contact aspiration.

Physicians typically rely on visual and tactile feedback to assess clot engagement to a clot retrieval device. In some treatments it can be difficult to determine whether a clot is engaged by a clot retrieval device with the present techniques.

### SUMMARY

In some examples, a clot removal assembly is disclosed for removing clot from a vessel. The clot removal assembly can include a delivery microcatheter, a cage structure configured to transform between a delivery configuration and an expanded pinching configuration, and a shaft in communication with the cage structure and substantially disposed within the delivery microcatheter. The delivery microcatheter can include: a distal portion including an elongate radiopaque indicator and a proximal portion. The elongate radiopaque indicator disposed on the distal portion of the delivery microcatheter aids the user in visualizing the pinching of a clot.

In some examples, the elongate radiopaque indicator can be configured to transform between a substantially straight non-indicating state and a substantially wavy indicating state responsive to an impingement of the cage structure on the delivery microcatheter.

In some examples, the elongate radiopaque indicator can be configured to transform between the substantially straight non-indicating state and the substantially wavy indicating state responsive to a proximally directed pulling force applied to the cage structure by the shaft.

In some examples, the elongate radiopaque indicator can be configured to transform between the substantially straight non-indicating state and the substantially wavy indicating state responsive to a distally directed pushing force applied to the microcatheter.

In some examples, the elongate radiopaque indicator can include a tungsten doped portion of the distal portion.

In some examples, the elongate radiopaque indicator includes a radiopaque coating.

In some examples, the cage structure can further include a distal section and a proximal spiral section. The distal section can include a first plurality of struts, and the proximal spiral section can include a second plurality of struts smaller than the first plurality of struts.

In some examples, a first end of the first plurality of struts abuts the second plurality of struts, and a second end of the first plurality of struts terminates in a plurality of atraumatic unions.

In some examples, the cage structure can include a proximal spiral section, a cylindrical body section distal of the proximal spiral section, and an open distal end. The proximal spiral section can be configured to pinch a clot between struts of the proximal spiral section.

In some examples, the distal portion of the delivery microcatheter can include a coiled core and the proximal portion of the delivery microcatheter can include a braided core.

In some examples, a method of treatment for an embolism is disclosed. The method can include deploying at least a part of a cage structure of a clot retrieval assembly across a clot and distal in relation to a catheter, applying a tension to a shaft attached to a proximal section of the cage structure, transforming an elongate radiopaque indicator disposed on a distal portion of the catheter from a first substantially straight non-indicating state to a second substantially wavy indicating state by pinching the clot between the cage structure and a distal end of the catheter, visualizing the substantially wavy indicating state of the elongate radiopaque indicator disposed on the distal end of the catheter, and extracting the clot, the cage structure and the catheter.

In some examples, the method can further include moving the cage structure to pin the clot between the cage structure and an inner wall of a blood vessel.

In some examples, the clot can be made up of a majority of fibrin.

In some examples, the method can include extracting the clot from a distal M2, M3, M4, A2-5, or P2-P5 vessel.

In some examples, visualizing the substantially wavy state can include using fluoroscopic imaging techniques.

In some examples, a catheter is disclosed. The catheter can include a proximal region extending over a majority of a length of the catheter, a distal region extending distally from the proximal region, and a radiopaque indicator extending over at least a portion of a length of the distal region. The catheter can be configured such that a compressive force applied at a distal end of the catheter results in a controlled movement of the distal region from a substantially straight uncompressed configuration to a wavy compressed configuration, and wherein the radiopaque indicator is configured to indicate a configuration of the distal region.

In some examples, the proximal region is configured to remain in a substantially straight configuration as the compressive force is applied at the distal end of the catheter and the distal region is moved to the wavy compressed configuration.

In some examples, the catheter can be configured such that the compressive force applied to a majority of the length of the catheter results in the proximal region remaining in a substantially straight configuration and the distal region moving to the wavy compressed configuration.

In some examples, the distal region can include a coiled support structure, and the proximal region includes a braided support structure.

In some examples, the catheter can be configured to deliver a clot retrieval device across a clot.

In some examples, the catheter can be configured to pinch the clot between a distal end of the catheter and the clot retrieval device.

Other aspects and features of the present disclosure will become apparent to those skilled in the pertinent art, upon reviewing the following detailed description in conjunction with the accompanying figures.

### BRIEF DESCRIPTION OF THE DRAWINGS

The above and further aspects of this disclosure are further discussed with the following description of the accompanying drawings, in which like numerals indicate like structural elements and features in various figures. The drawings are not necessarily to scale, emphasis instead being placed upon illustrating principles of the disclosure. The figures depict one or more implementations of the inventive devices, by way of example only, not by way of limitation. It is expected that those of skill in the art can conceive of and combining elements from multiple figures to better suit the needs of the user.

FIG. 1A illustrates a side view of a clot removal assembly with a micro-catheter in a non-indicating state according to this disclosure.

FIG. 1B illustrates a side view of the clot removal assembly illustrated in FIG. 1A with the micro-catheter in an indicating state according to this disclosure.

FIG. 2 is an isometric view of a cage structure according to this disclosure.

FIG. 3A illustrates a side view of a clot removal assembly in a delivery configuration according to this disclosure.

FIG. 3B illustrates a side view of the clot removal assembly illustrated in FIG. 3A with a microcatheter in an indicating state according to this disclosure.

FIG. 4 is an isometric view and a magnified cross-sectional view of a microcatheter according to this disclosure.

FIG. 5A is a cross-sectional view of a blood vessel and a clot and a clot removal assembly disposed therein according to this disclosure.

FIG. 5B is a cross-sectional view of a blood vessel and a clot and a clot removal assembly engaged to the clot according to this disclosure.

FIG. 6 is a flow diagram illustrating a method of treatment for an embolism, according to aspects of the present disclosure.

### DETAILED DESCRIPTION

The following detailed description is merely exemplary in nature and is not intended to limit the disclosure or the application and uses of the disclosure. Although the description of the disclosure is in many cases in the context of treatment of intracranial arteries, the disclosure may also be used in other body passageways as previously described.

The terms “distal” or “proximal” are used in the following description with respect to a position or direction relative to the treating physician. “Distal” or “distally” are a position distant from or in a direction away from the physician. “Proximal” or “proximally” or “proximate” are a position near or in a direction toward the physician.

As discussed herein, a “patient” or “subject” can be a human or any animal. It should be appreciated that an animal can be a variety of any applicable type, including, but not limited to, mammal, veterinarian animal, livestock animal or pet-type animal, etc. As an example, the animal can be a laboratory animal specifically selected to have certain characteristics similar to a human e.g., rat, dog, pig, monkey, or the like.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 99%.

When used herein, the terms “tubular” and “tube” are to be construed broadly and are not limited to a structure that is a right cylinder or strictly circumferential in cross-section or of a uniform cross-section throughout its length. For example, the tubular structure or system is generally illustrated as a substantially right cylindrical structure. However, the tubular system may have a tapered or curved outer surface without departing from the scope of the present invention.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

It must also be noted that, as used in the specification and the appended claims, the singular forms “a,” “an,” and “the” include plural referents unless the context clearly dictates otherwise. Ranges can be expressed herein as from “about” or “approximately” one particular value and/or to “about” or “approximately” another particular value. When such a range is expressed, other exemplary examples include from the one particular value and/or to the other particular value.

Accessing cerebral, coronary and pulmonary vessels involves the use of a number of commercially available products and conventional procedural steps. Access products such as guidewires, guide catheters, angiographic catheters and microcatheters are described elsewhere and are regularly used in catheter lab procedures. It is assumed in the descriptions below that these products and methods are employed in conjunction with the device and methods of this disclosure and do not need to be described in detail.

A common theme across many of the disclosed designs is an elongate radiopaque indicator 112 disposed on a delivery microcatheter 110 and configured to transform between a substantially straight non-indicating state 114 and a substantially wavy indicating state 115 responsive to an impingement of a cage structure 120 on the delivery microcatheter 110. This impingement can be the result the user pulling the cage structure proximally or advancing the delivery microcatheter distally while the cage structure 120 is held open (e.g. by a clot or other obstruction), inhibiting the cage structure 120 from being fully retracted into the microcatheter 110.

The cage structures 120 of the designs disclosed are desirably made from a material capable of recovering its shape automatically once released from a highly strained delivery configuration. A superelastic material such as Nitinol or an alloy of similar properties is particularly suitable. The material can be in many forms such as wire or strip or sheet or tube. A particularly suitable manufacturing process is to laser cut a Nitinol tube and then heat set and electropolish the resultant structure to create a framework of struts and connecting elements. This framework can be any of a huge range of shapes as disclosed herein and may be rendered visible under fluoroscopy through the addition of alloying elements (such as platinum) or through a variety of other coatings or marker bands.

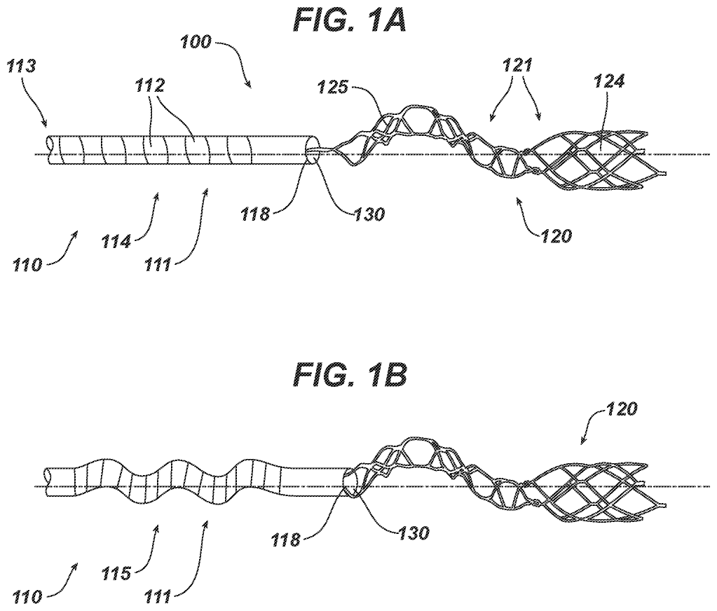

FIG. 1A is an illustration of one example clot removal assembly 100 for removing a clot 160 from a vessel 170. The clot removal assembly 100 can include a delivery microcatheter 110, a cage structure 120, and a shaft 130 in communication with the cage structure 120 and substantially disposed within the delivery microcatheter 110. The delivery microcatheter 110 can include a distal portion 111 and a proximal portion 113. The distal portion 111 includes an elongate radiopaque indicator 112. The proximal portion 113 can be manipulated to position the distal portion 111 within a patient.

In some examples, the elongate radiopaque indicator 112 can be configured to transform between a substantially straight non-indicating state 114, such as that shown in FIG. 1A, and a substantially wavy indicating state 115, such as that shown in FIG. 1B, responsive to an impingement of the cage structure 120 on the delivery microcatheter 110.

In some examples, elongate radiopaque indicator 112 can be configured to transform between the substantially straight non-indicating state 114 and the substantially wavy indicating state 115 responsive to a proximally directed pulling force applied to the cage structure 120 by the shaft 130.

In some examples, the elongate radiopaque indicator 112 can be configured to transform between the substantially straight non-indicating state 114 and the substantially wavy indicating state 115 responsive to a distally directed pushing force applied to the microcatheter 110.

In some examples, the elongate radiopaque indicator 112 can include a tungsten doped portion of the distal portion 111.

In some examples, the elongate radiopaque indicator 112 can include a radiopaque coating.

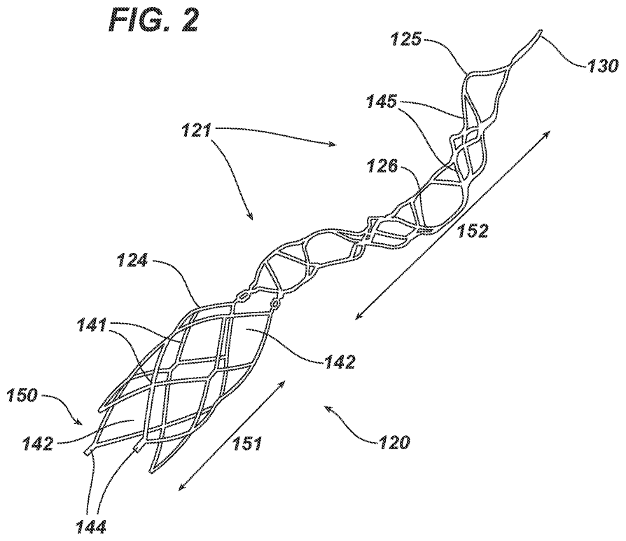

FIG. 2 is an isometric view of a cage structure 120. In some examples, the cage structure can include a distal section 124 and a proximal spiral section 125. The distal section 124 can include a first plurality of struts 141, and the proximal spiral section 125 can include a second plurality of struts 145 smaller than the first plurality of struts 141. The second plurality of struts 145 can be smaller than the first plurality of struts 141 by virtue of struts in the second plurality of struts 145 being shorting in length than struts of the first plurality of struts 141.

In some examples, a first end 142 of the first plurality of struts 141 abuts the second plurality of struts 145, and a second end 143 of the first plurality of struts 141 terminates in a plurality of atraumatic unions 144.

In some examples, the cage structure 120 can include a proximal spiral section 125, a cylindrical body section 124 distal of the proximal spiral section 125, and an open distal end 150. The proximal spiral section 125 can be configured to pinch a clot 160 between struts 145 of the proximal spiral section 125.

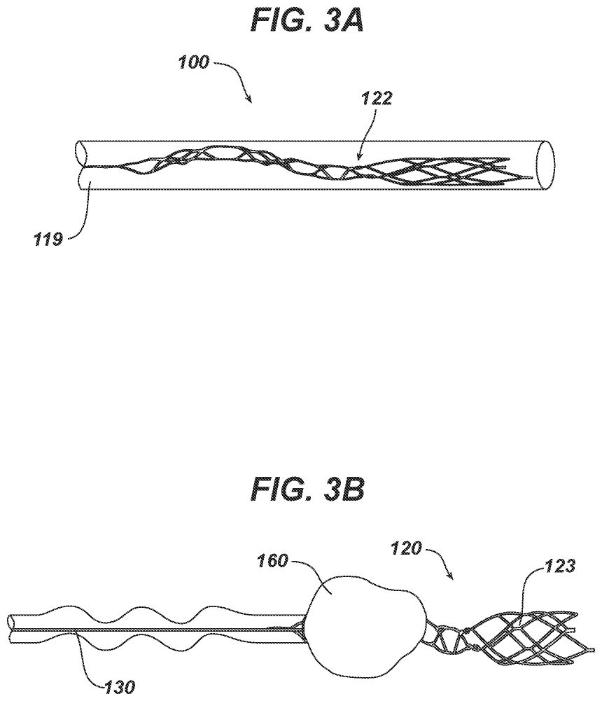

FIG. 3A illustrates a side view of a clot removal assembly 100 in a delivery configuration 122. FIG. 3B illustrates a side view of the clot removal assembly 100 illustrated in FIG. 3A with a microcatheter 110 in an indicating state. The cage structure 120 can be configured to transform between a delivery configuration 122 (FIG. 3A) and an expanded pinching configuration 123 (FIG. 3B).

As shown in FIG. 3A, in the delivery configuration 122, the cage structure 120 is disposed within the delivery microcatheter 110. In the delivery configuration 122, the first plurality of struts 141, the second plurality of struts 145, the proximal spiral section 125, and the cylindrical body section 124 can be generally aligned with the longitudinal axis of the delivery microcatheter 110.

As shown in FIG. 3B, in the expanded pinching configuration 123, the cage structure 120 can be completely or partially outside of the delivery microcatheter 110. The cage structure 120 can be translated along the longitudinal axis in relation to the delivery microcatheter 110 such that the cage structure 120 can take on various configurations between the delivery configuration 122 and the expanded pinching configuration 123. The clot 160 may partially or completely block the cage structure 120 from being retracted proximally into the delivery microcatheter 110. The distal portion 111 of the microcatheter 110 can have sufficient flexibility such that this blocking impingement between the delivery microcatheter 110, the clot 160, and the cage structure 120 can cause the radiopaque indicator 121 disposed at the distal portion 111 of the delivery microcatheter 112 to deform. As the distal portion 111 of the microcatheter 110 deforms, the radiopaque indicator 121 becomes wavy and thus take on the substantially wavy indicating state 115. As the cage structure 120 is retracted by the shaft 130 and impinging on the distal end of the microcatheter 110, the shaft 130 is under tension within the microcatheter 110 such that greater tension in the shaft 130 results in greater compressive force on the distal portion 111 of the microcatheter 110. The shaft 130 remains in a non-wavy state due to the proximally directed pulling force applied to it.

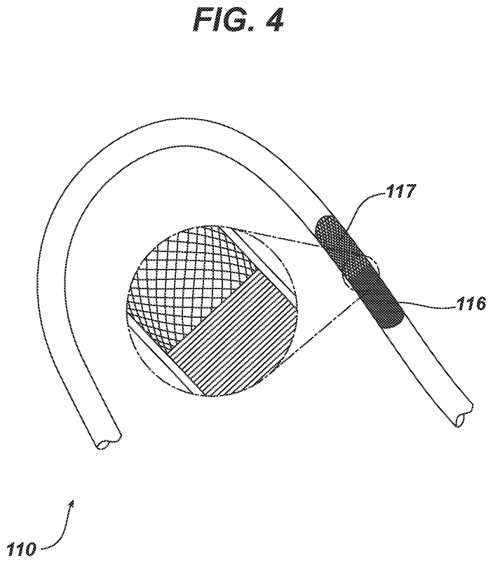

FIG. 4 is an isometric view and a magnified cross-sectional view of an example microcatheter 110. In some examples, the distal portion 111 of the delivery microcatheter 110 can include a coiled core 116 and the proximal portion 113 of the delivery microcatheter 110 can include a braided core 117. A radiopaque indicator similar to those disclosed and illustrated elsewhere herein, variations thereof, and alternatives thereto can be included in the distal portion of the microcatheter 110. The radiopaque indicator can be moved from a non-indicating state to an indicating state similar to as disclosed and illustrated elsewhere herein, variations thereof, and alternatives thereto.

In some examples, the coiled core 116 can provide the delivery microcatheter 110 with a level of flexibility that allows the user to better navigate tortuous distal vessels. In some examples, the braided core 117 of the proximal portion 113 can provide the delivery microcatheter 110 with a level of column strength that aids the user in navigating the tip of the microcatheter to the target anatomy. In some examples, flexibility of the distal portion 111 containing the coiled core coupled with the column strength of the proximal portion containing the braided core can aid in the transformation of the elongate radiopaque indicator 112 between the substantially straight non-indicating state 114 and the substantially wavy indicating state 115.

In some examples, the catheter 110 can include a proximal region 113 extending over a majority of a length of the catheter, a distal region 111 extending distally from the proximal region 113, and a radiopaque indicator 112 extending over at least a portion of a length of the distal region 111. The catheter 110 can be configured such that a compressive force applied at a distal end 118 of the catheter results in a controlled movement of the distal region from a substantially straight uncompressed configuration 114 to a wavy compressed configuration 115, and wherein the radiopaque indicator 112 is configured to indicate a configuration of the distal region 111.

In some examples, the proximal region 113 is configured to remain in a substantially straight configuration 119 as the compressive force is applied at the distal end 118 of the catheter and the distal region is moved to the wavy compressed configuration 115.

In some examples, the catheter 110 can be configured such that the compressive force applied to a majority of the length of the catheter results in the proximal region remaining in a substantially straight configuration 114 and the distal region moving to the wavy compressed configuration 115.

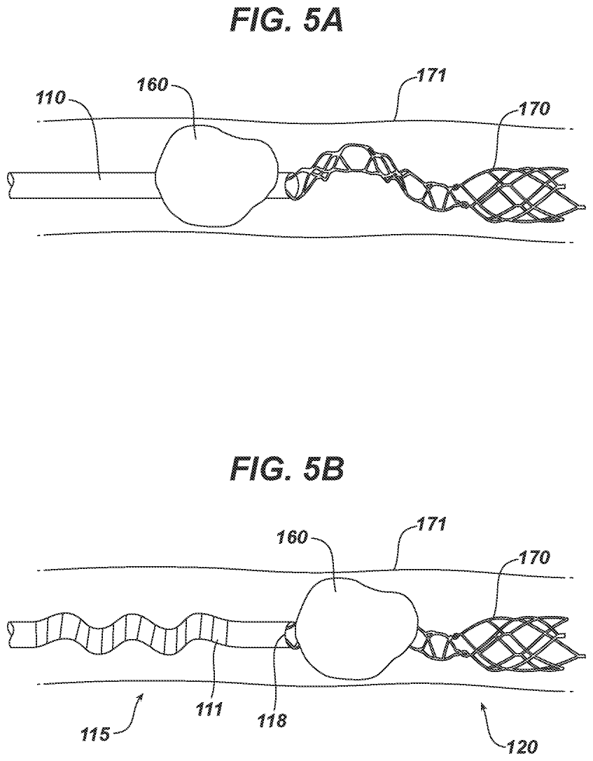

FIG. 5A is a cross-sectional view of a blood vessel 171, a clot 160, and a clot removal assembly 100 disposed therein. In some examples, the catheter 110 can be configured to deliver the clot retrieval device 120 across the clot 160.

FIG. 5B is a cross-sectional view of the blood vessel 171, the clot 160, and the clot removal assembly 100 illustrated in FIG. 5A with the clot removal assembly 100 engaged to the clot 160. In some examples, the spiral section 125 of the cage structure 120 can be sized to wind within the blood vessel 171 against walls of the blood vessel 171. The spiral section 125 can push the clot 160 into the blood vessel wall, pinning the clot 160 so that the clot is inhibited from moving distally through the vessel 171. The microcatheter 110 can be advanced distally, causing the struts of the spiral section 125 to move toward each other, like tweezers.

The clot 160 can have a firm portion that can be pinched between the tip of the catheter 110 and the struts of the spiral section 125 of the cage structure 120. The pinch can be achieved by forwarding the microcatheter 110 or intermediate catheter over the cage structure 120 until a portion of the clot 160 is compressed between the tip of the catheter 110 and a crown or strut on the proximal portion of the cage structure 120. This pinch facilitates removal of the clot 160 as it increases the grip of the cage structure 120 on the clot, particularly fibrin rich clots. Proximal retraction of the pinched clot may elongate the clot 160 reducing the dislodgement force by pulling the clot 160 away from the vessel wall during the dislodgement process.

In some examples, the distal section 124 of the cage structure 120 can have a barrel shape sized to expand to occlude the vessel 171 so that the clot 160 is inhibited from moving distally as the clot 120 and assembly 100 are moved proximally to extract the clot 160. In some examples, distal section 124 may further be configured to expand through a soft portion of the clot 160 to further engage the clot 160.

The pinching of the clot between the catheter 110 and the cage structure 120 can cause the radiopaque indicator 112 to transform between a substantially straight non-indicating state 114, such as that shown in FIG. 1A, and a substantially wavy indicating state 115, such as that shown in FIG. 1B, responsive to an impingement of the cage structure 120 on the delivery microcatheter 110.

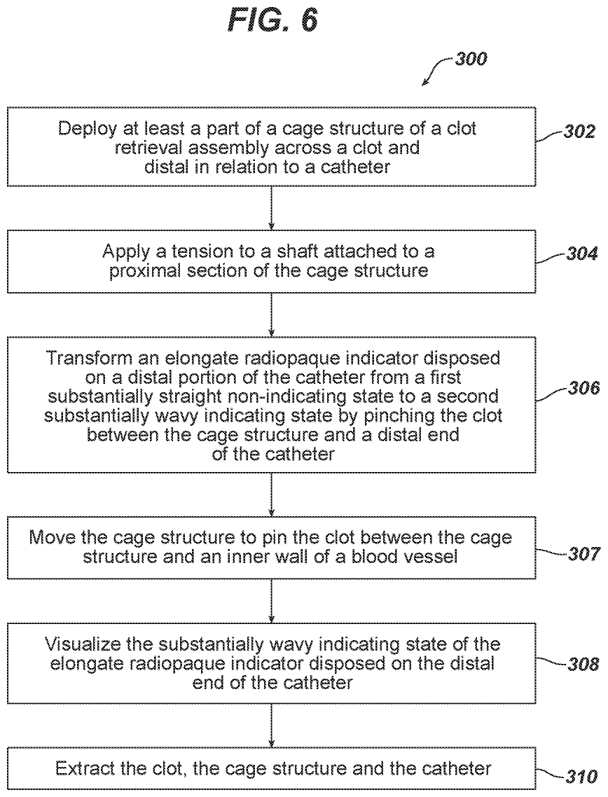

FIG. 6 shows a method 300 of treatment for an embolism as disclosed herein. The method steps in FIG. 6 can be implemented by any of the example means described herein or by similar means, as will be appreciated.

At block 302, the method 300 can include deploying at least a part of a cage structure of a clot retrieval assembly across a clot and distal in relation to a catheter. The cage structure can be configured similarly to an example cage structure 120 disclosed herein, variations thereof, and alternatives thereto as understood by a person skilled in the pertinent art. The clot retrieval assembly can be configured to an example clot retrieval assembly 100 disclosed herein, variations thereof, and alternatives thereto as understood by a person skilled in the pertinent art. The cage structure can be deployed similarly to as illustrated in FIG. 1B, 3B, 5B, as otherwise disclosed herein, variations thereof, and alternatives thereto as understood by a person skilled in the pertinent art.

At block 304, the method 300 can include applying a tension to a shaft attached to a proximal section of the cage structure. The shaft can be configured similarly to an example shaft 130 disclosed herein, variations thereof, and alternatives thereto as understood by a person skilled in the pertinent art. The proximal section of the cage structure can be configured similarly to an example proximal section 125 of a cage structure 120 disclosed herein, variations thereof, and alternatives thereto as understood by a person skilled in the pertinent art.

At block 306, the method 300 can include transforming an elongate radiopaque indicator disposed on a distal portion of the catheter from a first substantially straight non-indicating state to a second substantially wavy indicating state by pinching the clot between the cage structure and a distal end of the catheter. The elongate radiopaque maker can be configured similarly to an example radiopaque indicator 112 disclosed herein, variations thereof, and alternatives thereto as understood by a person skilled in the pertinent art. The distal portion of the catheter can be configured similarly to an example distal portion 111 of a catheter 110 disclosed herein, variations thereof, and alternatives thereto as understood by a person skilled in the pertinent art. The straight non-indicating state can be similar to non-indicating state 114, similar as illustrated in FIG. 1A, 3A, or 5A, as otherwise disclosed herein, variations thereof, and alternatives thereto as understood by a person skilled in the pertinent art. The second substantially wavy indicating state can be similar to indicating state 115, similar to as illustrated in FIG. 1B, 3B, or 5B, as otherwise disclosed herein, variations thereof, and alternatives thereto as understood by a person skilled in the pertinent art. The clot can be pinched to the distal end of the catheter similar to as illustrated in FIG. 5B, as otherwise disclosed herein, variations thereof, and alternatives thereto as understood by a person skilled in the pertinent art.

At block 307, the method 300 can include moving the cage structure to pin the clot between the cage structure and an inner wall of a blood vessel. The clot can be pinned by the cage structure as disclosed herein, variations thereof, and alternatives thereto as understood by a person skilled in the pertinent art.

At block 308, the method can include visualizing the substantially wavy indicating state of the elongate radiopaque indicator disposed on the distal end of the catheter. In some examples, the visualizing the substantially wavy state can include using fluoroscopic imaging techniques.

At block 310, the method can include extracting the clot, the cage structure, and the catheter.

In some examples, the clot can be made up of a majority of fibrin.

In some examples, the method 300 can include extracting the clot from a distal M2, M3, M4, A2-5, or P2-P5 vessel. The size of any of these vessels is known in the art and it will be appreciated by those skilled in the pertinent art that the components of the clot retrieval assembly can be sized appropriately to achieve the goal of removing the clot from these vessels.

In some examples, the cage structure 120 can further include cells, struts, and a variety of shapes and designs configured for pinching fibrin rich clots, including those described in U.S. Pat. Nos. 10,292,723; 10,363,054; 10,617,435; 11,253,278; and 11,147,572, each of which are incorporated by reference in their entirety as if set forth verbatim herein. Compression of the clot by the cage structure 120, including by the cylindrical body section 124, can alter the clot properties and make the clot less amenable to retrieval by making it firmer and “stickier” similar to as described in WO2012/120490A, the entire contents of which are herein incorporated by reference.

In some examples, distal advancement of the microcatheter 110 relative to the cage structure 120 can compress the clot 160 between the distal end 118 of the microcatheter 110 and the cage structure 120, increasing the pinch on the clot 160 and the security of the trapped clot segment. The user may feel this pinch as a resistance and stop advancing the microcatheter 110, the user may advance the delivery microcatheter 110 a fixed distance over the shaft 130 for example 30% to 50% of the shaft length, or the user may visualize the substantially wavy indicating state 115 of the radiopaque indicator 112 to confirm pinching of the clot 160 before retracting the clot removal assembly 100.

The relative tension between the cage structure 120 and the delivery microcatheter 110 needs to be maintained to ensure the pinch between the clot removal assembly 100 and the clot 160 does not deteriorate. By retracting the clot 160 and the clot removal assembly 100 together, the occlusive clot 160 can be dislodged and retracted back into a proximally located access guide catheter not shown or introducer sheath not shown and be removed from the patient.

Shaft 130 may be a tapered wire shaft, and may be made of stainless steel, MP35N, Nitinol or other material of a suitably high modulus and tensile strength.

The elongate radiopaque indicator 112 can be rendered radiopaque through the addition of a filler material such as tungsten or barium sulphate. However, other radiopaque materials are contemplated, including but not limited to Bismuth SubCarbonate, Barium OxyChloride, Gold, Platinum, Iridium, Tantalum or an alloy of any of these materials.

A range of designs are envisaged for each of these elements as described throughout this document, and it is intended that any of these elements can be used in conjunction with any other element, although to avoid repetition they are not shown in every possible combination.

In some examples, the clot 160 can be made up of a majority of fibrin.

In some examples, the method 300 can include extracting the clot 160 from a distal M2, M3, M4, A2-5, or P2-P5 vessel.

In some examples, the visualizing the substantially wavy state 115 can include using fluoroscopic imaging techniques.

In some examples, a catheter 110 is disclosed. The catheter 110 can include a proximal region 113 extending over a majority of a length of the catheter 110, a distal region 111 extending distally from the proximal region 113, and a radiopaque indicator 112 extending over at least a portion of a length of the distal region 111. The catheter 110 can be configured such that a compressive force applied at a distal end 118 of the catheter 110 results in a controlled movement of the distal region 111 from a substantially straight uncompressed configuration 114 to a wavy compressed configuration 115, and wherein the radiopaque indicator 112 is configured to indicate a configuration of the distal region 111.

In some examples, the proximal region 113 is configured to remain in a substantially straight configuration 119 as the compressive force is applied at the distal end 118 of the catheter 110 and the distal region 111 is moved to the wavy compressed configuration 115.

In some examples, the catheter 110 can be configured such that the compressive force applied to a majority of the length of the catheter 110 results in the proximal region 113 remaining in a substantially straight configuration 119 and the distal region moving to the wavy compressed configuration 115.

In some examples, the distal region 111 can include a coiled support structure 116, and the proximal region 113 includes a braided support structure 117.

In some examples, the catheter 110 can be configured to deliver a clot retrieval device 120 across a clot 160.

In some examples, the catheter 110 can be configured to pinch the clot 160 between a distal end 118 of the catheter 110 and the clot retrieval device 120.

Other aspects and features of the present disclosure will become apparent to those of skill in the pertinent art, upon reviewing the following detailed description in conjunction with the accompanying figures.

In describing examples, terminology is resorted to for the sake of clarity. It is intended that each term contemplates its broadest meaning as understood by those skilled in the pertinent art and includes all technical equivalents that operate in a similar manner to accomplish a similar purpose. It is also to be understood that the mention of one or more steps of a method does not preclude the presence of additional method steps or intervening method steps between those steps expressly identified. Steps of a method can be performed in a different order than those described herein without departing from the scope of the disclosed technology. Similarly, it is also to be understood that the mention of one or more components in a device or system does not preclude the presence of additional components or intervening components between those components expressly identified.

The descriptions contained herein are examples of the disclosure and are not intended in any way to limit the scope of the disclosure. While particular examples of the present disclosure are described, various modifications to devices and methods can be made without departing from the scope and spirit of the disclosure. For example, while the examples described herein refer to particular components, the disclosure includes other examples utilizing various combinations of components to achieve a described functionality, utilizing alternative materials to achieve a described functionality, combining components from the various examples, combining components from the various example with known components, etc. The disclosure contemplates substitutions of component parts illustrated herein with other well-known and commercially available products. To a person skilled in the pertinent art, these modifications are often apparent and are intended to be within the scope of the claims which follow.

What is claimed is:

1. A clot removal assembly, comprising:
a delivery microcatheter comprising:
a distal portion comprising an elongate radiopaque indicator, and
a proximal portion;
a cage structure configured to transform between a delivery configuration and an expanded pinching configuration; and
a shaft in communication with the cage structure and substantially disposed within the delivery microcatheter,
wherein the elongate radiopaque indicator is configured to transform between a substantially straight non-indicating state and a substantially wavy indicating state responsive to an impingement of the cage structure on the delivery microcatheter.

2. The clot removal assembly of claim 1, wherein the elongate radiopaque indicator is configured to transform between the substantially straight non-indicating state and the substantially wavy indicating state responsive to a proximally directed pulling force applied to the cage structure by the shaft.

3. The clot removal assembly of claim 1, wherein the elongate radiopaque indicator is configured to transform between the substantially straight non-indicating state and the substantially wavy indicating state responsive to a distally directed pushing force applied to the delivery microcatheter.

4. The clot removal assembly of claim 1, wherein the elongate radiopaque indicator comprises a tungsten doped portion of the distal portion.

5. The clot removal assembly of claim 1, wherein the elongate radiopaque indicator comprises a radiopaque coating.

6. The clot removal assembly of claim 1, wherein the cage structure further comprises a distal section and a proximal spiral section, the distal section comprising a first plurality of struts and the proximal spiral section comprising a second plurality of struts smaller than the first plurality of struts.

7. The clot removal assembly of claim 6, wherein a first end of the first plurality of struts abuts the second plurality of struts and a second end of the first plurality of struts terminates in a plurality of atraumatic unions.

8. The clot removal assembly of claim 1, wherein the cage structure comprises a proximal spiral section, a cylindrical body section distal of the proximal spiral section, and an open distal end, wherein the proximal spiral section is configured to pinch a clot between struts of the proximal spiral section.

9. The clot removal assembly of claim 1, wherein the distal portion of the delivery microcatheter comprises a coiled core and the proximal portion of the delivery microcatheter comprises a braided core.

10. A method of treatment for an embolism, the method comprising:
deploying at least a part of a cage structure of a clot retrieval assembly across a clot and distal in relation to a catheter;
applying a tension to a shaft attached to a proximal section of the cage structure;
transforming an elongate radiopaque indicator disposed on a distal portion of the catheter from a first substantially straight non-indicating state to a second substantially wavy indicating state by pinching the clot between the cage structure and a distal end of the catheter;
visualizing the second substantially wavy indicating state of the elongate radiopaque indicator disposed on the distal end of the catheter; and
extracting the clot, the cage structure, and the catheter.

11. The method of claim 10, further comprising:
moving the cage structure to pin the clot between the cage structure and an inner wall of a blood vessel.

12. The method of claim 10, wherein the clot comprises a majority of fibrin.

13. The method of claim 12, further comprising:
extracting the clot from a distal M2, M3, M4, A2-5, or P2-P5 vessel.

14. The method of claim 10, wherein the visualizing the second substantially wavy state comprises using fluoroscopic imaging techniques.

15. A catheter comprising:
a proximal region extending over a majority of a length of the catheter;
a distal region extending distally from the proximal region; and
a radiopaque indicator extending over at least a portion of a length of the distal region,
wherein the catheter is configured such that a compressive force applied at a distal end of the catheter results in a controlled movement of the distal region from a substantially straight uncompressed configuration to a wavy compressed configuration, and
wherein the radiopaque indicator is configured to indicate a configuration of the distal region.

16. The catheter of claim 15, wherein the proximal region is configured to remain in a substantially straight configuration as the compressive force is applied at the distal end of the catheter and the distal region is moved to the wavy compressed configuration.

17. The catheter of claim 15, wherein the catheter is configured such that the compressive force applied to a majority of the length of the catheter results in the proximal region remaining in a substantially straight configuration and the distal region moving to the wavy compressed configuration.

18. The catheter of claim 15,
wherein the distal region comprises a coiled support structure, and
wherein the proximal region comprises a braided support structure.

19. The catheter of claim 15, wherein the catheter is configured to deliver a clot retrieval device across a clot.

20. The catheter of claim 19, wherein the catheter is configured to pinch the clot between a distal end of the catheter and the clot retrieval device.
